(19) United States
(12) Patent Application Publication
(10) Pub. No.: US 2010/0056860 A1
(43) Pub. Date: Mar. 4, 2010

(54) Capsule endoscope with metal-insert molded contacts

(76) Inventors: Shih-Chieh Lu, Hsinchu (TW); Chun-Ming Lin, Hsinchu (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/461,390

(22) Filed: Aug. 11, 2009

(30) Foreign Application Priority Data
Aug. 26, 2008 (TW) 97132607

(51) Int. Cl. A61B 1/00

(52) U.S. Cl. 600/101

(57) ABSTRACT

A capsule endoscope for observing the conditions inside a patient's digestive tract system includes a capsular housing, a substrate, and a plurality of metal-insert molded contacts. The capsular housing defines a sealed inner space and has an outer surface. The substrate is arranged in the sealed inner space of the capsular housing and is mounted with at least an electronic element provided thereon. The metal-insert molded contacts are embedded in the capsular housing at predetermined positions thereof and each includes a contact base and an exposed contact portion formed on the contact base. The contact bases each have a connecting section extended into the sealed inner space of the capsular housing to electrically connect to the substrate. The exposed contact portions each have a front surface exposed from the outer surface of the capsular housing for contacting with a signal contact correspondingly provided on a capsule holder that holds the capsular housing thereto.

### FIELD OF THE INVENTION

The present invention relates to a capsule endoscope, and more particularly to a capsule endoscope with metal-insert molded contacts and capsule endoscope capable of sensing external environmental signals.

### BACKGROUND OF THE INVENTION

Medically, an endoscope is mainly used to examine a patient's digestive tract system. By moving the endoscope into the patient's digestive tract, it is able to directly obtain image signals showing the conditions inside the patient's digestive tract to assist in the medical diagnosis. Currently, an electronic endoscope is one of the most commonly adopted endoscope systems.

Taiwan Patent No. 200810727 discloses an endoscope device for acquiring at least one image and sending the acquired image to an electronic device for display. The endoscope device includes a light source module, an image pickup lens, a light sensing element, a circuit board, a flexible flat cable, and a connecting module. The light source module emits a light beam to illuminate the environment outside the endoscope. The image pickup lens collects light reflected from an external object to form an optical image, based on which the light sensing element generates electronic signals. The electronic signals are processed and then shown on a display.

The electronic endoscope device is expensive and tends to cause cross infection when being repeatedly used. Further, the electronic endoscope device can only be used to examine the esophagus, stomach, and large intestine. To overcome the drawbacks of the electronic endoscope device, there is developed a capsule endoscope. When necessary, the capsule connected to a front end of the endoscope device can be separated therefrom via a releasing mechanism and therefore overcome the problem of limited observation range as found with the current electronic endoscope.

Currently, to manufacture the capsule endoscope, a mold must be formed for molding a plurality of half-housings. Thereafter, different electronic components, image pickup modules and other related devices are arranged in the half-housings before the corresponding half-housings are joined to form a complete capsular housing for the capsule endoscope. The above manufacturing process involves complicated steps and thereby requires increased manufacturing cost. Further, the capsular housing so produced has relatively low structural strength, and any joints or burrs formed in the process of joining the half-housings to form the capsular housing would very possibly cause discomfort to the patient once the defective capsule endoscope is put in the patient's digestive tract system for examination. In some worse conditions, the defective capsule endoscope would even hurt the patient's digestive tract.

Conventionally, the capsule endoscope is simply used to acquire images in the patient's digestive tract. When it is necessary to further diagnose any other illness related to the patient's digestive tract system, other examinations must be additional conducted using other suitable medical instruments.

### SUMMARY OF THE INVENTION

It is therefore a primary object of the present invention to provide a capsule endoscope with metal-insert molded contacts, which is formed using metal insert injection molding, so that a capsular housing of the capsule endoscope and electronic components enclosed in the capsular housing are integrally formed.

Another object of the present invention is to provide a capsule endoscope capable of sensing external environmental signals, so that the capsule endoscope can be used to sense the various signals around a position in a patient's digestive tract system at where the capsule endoscope is located.

To achieve the above and other objects, a capsule endoscope is provided. According to an embodiment of the present invention, the capsule endoscope includes a capsular housing, a substrate, and a plurality of metal-insert molded contacts. The capsular housing defines a sealed inner space and has an outer surface. The substrate is arranged in the sealed inner space of the capsular housing and is mounted with at least an electronic element provided thereon. The metal-insert molded contacts are embedded in the capsular housing at predetermined positions thereof, and each of the metal-insert molded contacts includes a contact base and an exposed contact portion exposed from the capsular housing. The contact base includes a connecting section radially extended into the sealed inner space of the capsular housing to electrically connect to the substrate. The exposed contact portion is formed on the contact base and has a front surface exposed from the outer surface of the capsular housing. Signal contacts of a capsule holder correspondingly connect to the exposed contact portions of the metal-insert molded contacts.

In another embodiment, the capsule endoscope capable of sensing external environmental signals includes a capsular housing, a substrate, and a plurality of metal-insert molded contacts. The capsular housing defines a sealed inner space and has an outer surface. The substrate is arranged in the sealed inner space of the capsular housing and has a sensing circuit provided thereon. The metal-insert molded contacts are embedded in the capsular housing at predetermined positions thereof, and each of the metal-insert molded contacts includes a contact base and an exposed contact portion exposed from the capsular housing. The contact base includes a connecting section radially extended into the sealed inner space of the capsular housing to electrically connect to the substrate. The exposed contact portion is formed on the contact base and has a front surface exposed from the outer surface of the capsular housing. A high impedance exists among the metal-insert molded contacts, and the metal-insert molded contacts are able to contact and sense external environmental signals around the position at where the capsular housing is located. The sensed external environmental signals are sent to the sensing circuit on the substrate via the exposed contact portions and the contact bases of the metal-insert molded contacts.

With the capsular housing thereof being integrally formed by metal insert injection molding, the capsule endoscope of the present invention can be manufactured with simplified process at largely reduced cost. And since the capsular housing is integrally formed without the risk of cracking or breaking, the capsular housing is not subject to invasion by the body fluids in the patient's digestive tract system and is therefore safe for use. In addition to the image recording function, the metal-insert molded contacts on the capsule endoscope can sense external various signals around the position at where the capsule endoscope is located, such as the temperature, the pressure value, and the pH value and any bio-signals of inside the patient's digestive tract system. Therefore, with the capsule endoscope with metal-insert molded contacts according to the present invention, it is not necessary for the patient to accept different examinations using different testing devices, and the time needed to complete the examinations can be shortened, allowing the doctor to diagnose and take proper medical treatment within the first time point.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a perspective view of a capsule endoscope with metal-insert molded contacts according to a first embodiment of the present invention;

FIG. 2 is a cross sectional view taken along line 2-2 of FIG. 1;

FIG. 3 is a sectional view taken along line 3-3 of FIG. 2;

FIG. 4 is an exploded perspective view showing the capsule endoscope of FIG. 1 separated from a capsule holder;

FIG. 5 is an assembled view of FIG. 4 viewed from another angle;

FIG. 6 is a perspective view of a capsule endoscope according to a second embodiment of the present invention;

FIG. 7 schematically shows the capsule endoscope according to the second embodiment of the present invention moving through a patient's digestive tract system; and

FIG. 8 is a functional block circuit diagram of the present invention.

### DETAILED DESCRIPTION OF THE PREFERRED EMBODIMENTS

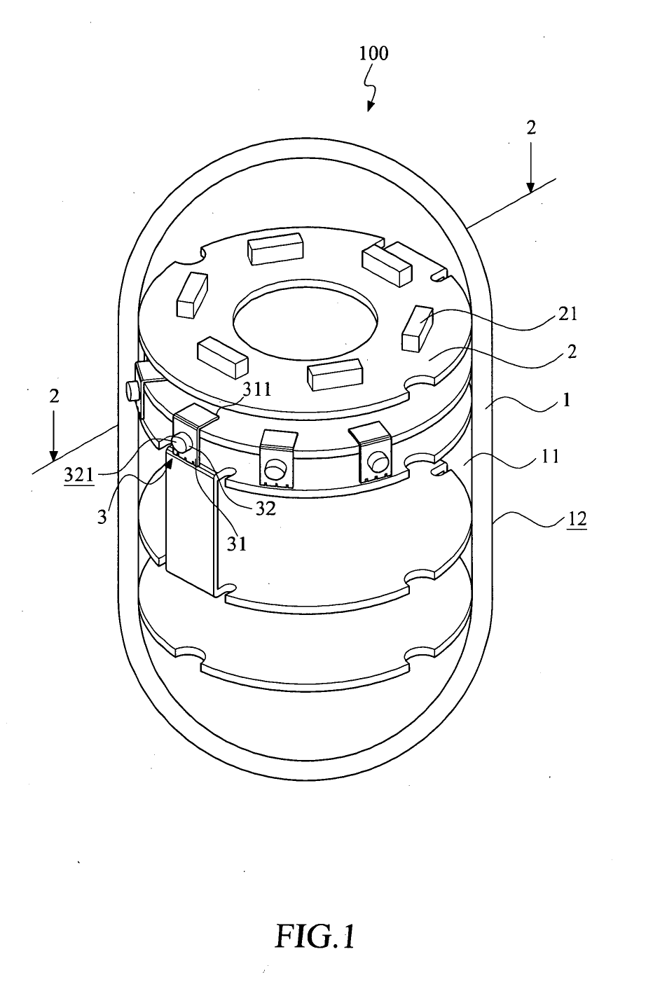

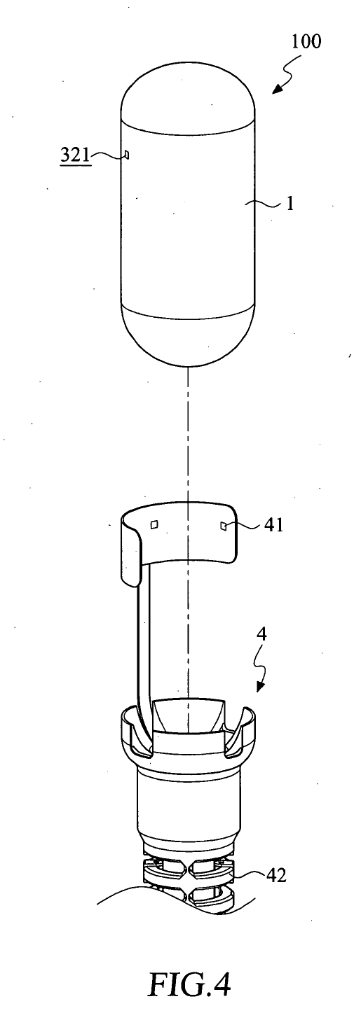

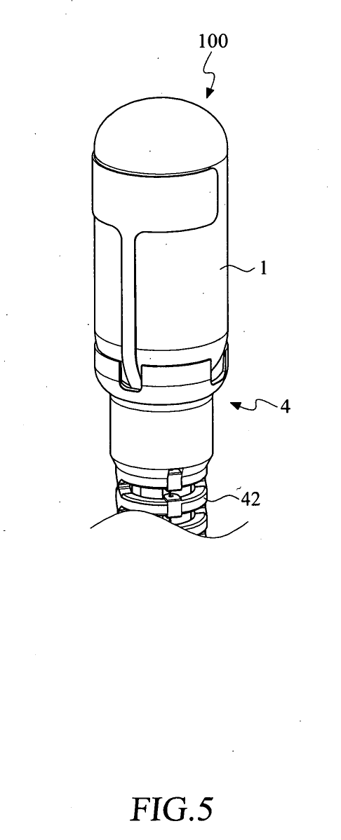

Please refer to FIG. 1 that is a perspective view of a capsule endoscope according to a first embodiment of the present invention. In the first embodiment, the capsule endoscope 100 provided with metal-insert molded contacts is used to observe the internal conditions of the patient's digestive tract system. As shown, the capsule endoscope 100 includes a capsular housing 1, a substrate 2, and a plurality of metal-insert molded contacts 3. The capsular housing 1 defines a sealed inner space 11 and has an outer surface 12. The substrate 2 is arranged in the sealed inner space 11 of the capsular housing 1 and has circuit elements 21 provided thereon. The metal-insert molded contacts 3 are embedded in the capsular housing 1 at predetermined positions and each include a contact base 31 and an exposed contact portion 32. The contact base 31 includes a connecting section 311 radially extending into the sealed inner space 11 of the capsular housing 1 to electrically connect to the substrate 2. The exposed contact portion 32 is formed on the contact base 31 and has a front surface 321 exposed from the outer surface 12 of the capsular housing 1. The capsule endoscope 100 can be held in a capsule holder 4 with the exposed front surfaces 321 of the exposed contact portions 32 contacting with a signal contact 41 correspondingly provided on the capsule holder 4 as shown in FIGS. 4 and 5. The metal-insert molded contacts 3 each can be formed from a metal sheet. Alternatively, the metal-insert molded contacts 3 each can be a flexible circuit board.

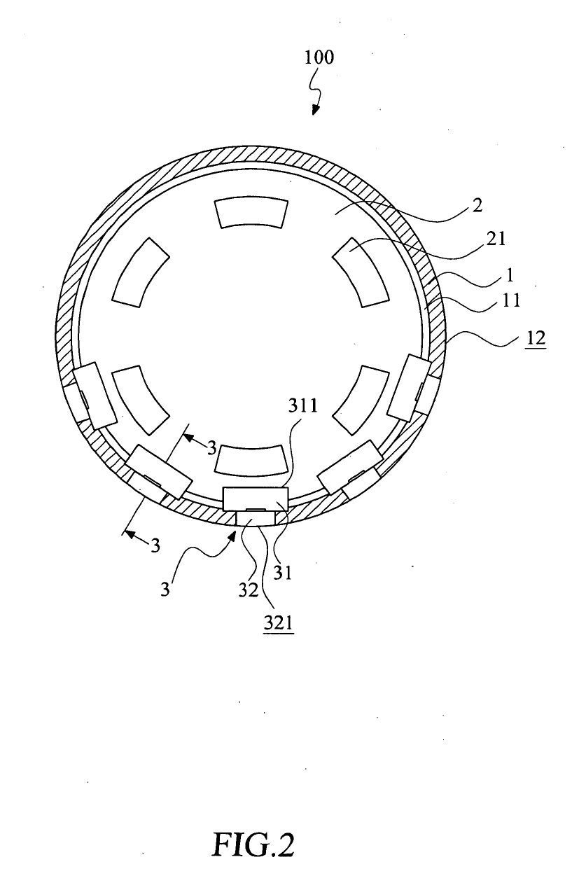

Please refer to FIG. 2 that is a cross-sectional view taken along line 2-2 of FIG. 1. As shown, the substrate 2 is arranged in the sealed inner space 11 of the capsular housing 1 at a predetermined position thereof and has circuit elements 21 provided thereon. The circuit elements 21 are radially arranged on the substrate 2 relative to a center of the substrate 2. The metal-insert molded contacts 3 are separately embedded in the capsular housing 1 within a predetermined sector position relative to the center of the substrate 2, such that the connecting sections 311 of the contact bases 31 of the metal-insert molded contacts 3 are radially extended from the capsular housing 1 into the sealed inner space 11 to electrically connect to the substrate 2. The exposed contact portions 32 of the metal-insert molded contacts 3 are formed on the contact bases 31 with the front surfaces 321 of the exposed contact portions 32 exposed from the outer surface 12 of the capsular housing 1. The outer surface 12 of the capsular housing 1 is a regular curved surface, and the front surfaces 321 of the exposed contact portions 32 are flush with the outer surface 12 of the capsular housing 1 to form a curve surface to prevent the capsule endoscope 100 with metal-insert molded contacts 3 from scratching and injuring a patient's gastric wall or intestinal wall. In the illustrated first embodiment, there is a high impedance between the metal-insert molded contacts 3 and the substrate 2 to prevent electric current in the circuit elements 21 on the substrate 2 from leaking via the metal-insert molded contacts 3 to produce electric pulse, which would adversely affect the patient's normal nerve conduction.

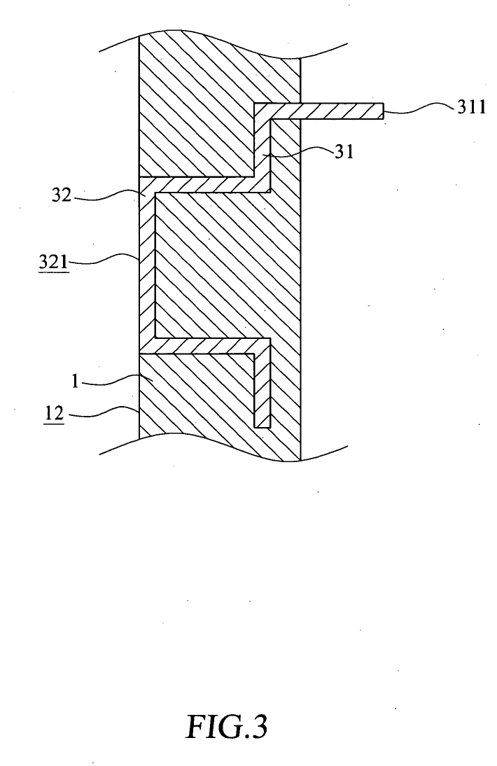

FIG. 3 is a sectional view taken along line 3-3 of FIG. 2. As shown, the metal-insert molded contact 3 is embedded in the capsular housing 1 with the connecting section 311 of the contact base 31 extending into the sealed inner space 11 of the capsular housing 1. The exposed contact portion 32 is formed on the contact base 31 with the front surface 321 of the exposed contact portion 32 exposing from but flushing with the outer surface 12 of the capsular housing 1. In the illustrated first embodiment, the exposed contact portion 32 is raised from the contact base 31 of the metal-insert molded contact 3 by way of stamping. However, it is understood the exposed contact portion 32 can be formed on the contact base 31 using other different ways, such as riveting. Further, a layer of metal material, such as gold or other conductive material, can be coated on the metal-insert molded contacts 3, so that the front surfaces 321 of the exposed contact portions 32 exposed from the outer surface 12 of the capsular housing 1 are protected against corrosion. In the instant embodiment, the contact base 31 of the metal-insert molded contact 3 is an L-shaped sheet member.

FIGS. 4 and 5 are exploded and assembled perspective views, respectively, showing the capsule endoscope 100 and a capsule holder 4 therefor. As shown, the capsule endoscope 100 can be held to an end of the capsule holder 4. The capsule holder 4 has a signal contact 41 located between the capsule holder 4 and the metal-insert molded contacts 3 to correspondingly connect to the exposed front surfaces 321 of the exposed contact portions 32. When the capsule endoscope 100 is firmly held to the end of the capsule holder 4 with the signal contact 41, the capsule endoscope 100 can be extended into the patient's digestive tract system through proper manipulation of a flexible tube 42 connected to another end of the capsule holder 4 for observing an internal conditions of the patient's digestive tract system.

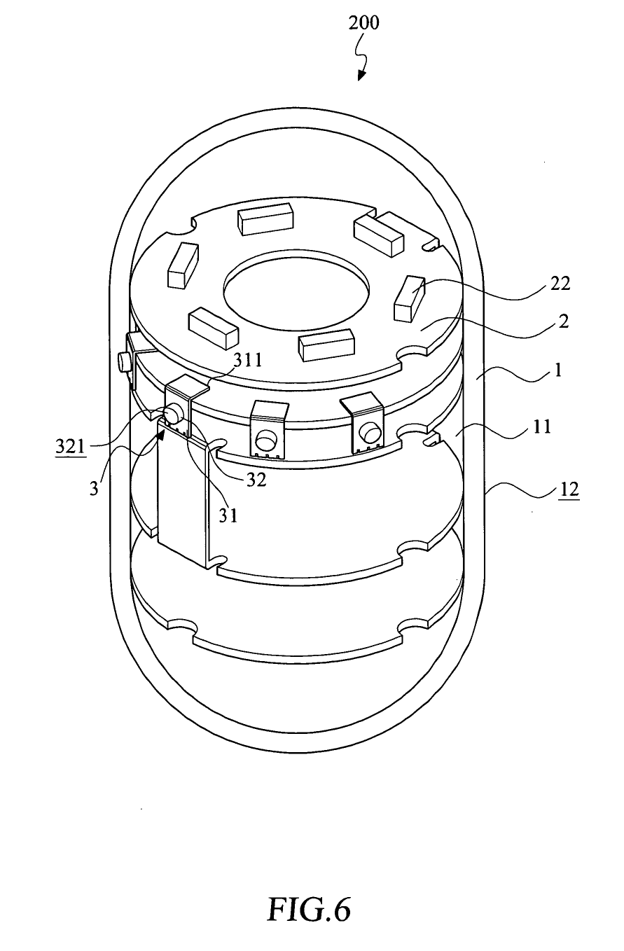

Please refer to FIG. 6 that is a perspective view of a capsule endoscope, which is capable of sensing external environmental signals, according to a second embodiment of the present invention. For the purpose of conciseness and clarity, the capsule endoscope capable of sensing external environmental signals according to a second embodiment of the present invention is also briefly referred to as the capsule endoscope and denoted by reference numeral 200 herein. As shown, the capsule endoscope 200 includes a capsular housing 1, a substrate 2, and a plurality of metal-insert molded contacts 3. The capsular housing 1 defines a sealed inner space 11 and has an outer surface 12. The substrate 2 is arranged in the sealed inner space 11 of the capsular housing 1 at a predetermined position thereof and has a sensing circuit 22 provided thereon. The metal-insert molded contacts 3 are embedded in the capsular housing 1 at predetermined positions thereof, and each include a contact base 31 and an exposed contact portion 32. The contact base 31 includes a connecting section 311 radially extending into the sealed inner space 11 of the capsular housing 1 to electrically connect to the substrate 2. The exposed contact portion 32 is formed on the contact base 31 and has a front surface 321 exposed from the outer surface 12 of the capsular housing 1 for sensing external environmental signals S around a position at where the capsule housing 1 is located. The sensed external environmental signals S are sent to the sensing circuit 22 on the substrate 2 via the exposed contact portions 32 and the contact bases 31 of the metal-insert molded contacts 3. The metal-insert molded contacts 3 each can be formed from a metal sheet. Alternatively, the metal-insert molded contacts 3 each can be a flexible circuit board.

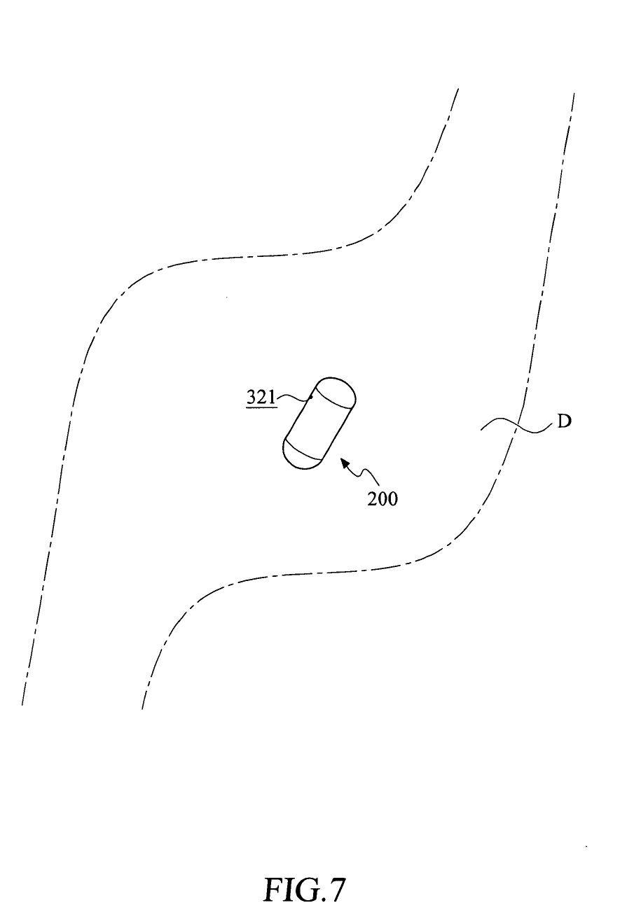

FIG. 7 shows the capsule endoscope 200 according to the second embodiment of the present invention moving through a patient's stomach D. As shown, the capsule endoscope 200 senses external environmental signals S using the front surfaces 321 of the exposed contact portions 32 exposed from the outer surface 12 of the capsular housing 1. Since the capsular housing 1 is located in the patient's stomach D, the external environmental signals S sensed by the exposed front surfaces 321 of the exposed contact portions 32 can be any one or more of a temperature value, a pressure value, and a pH value. And, the sensed external environmental signals are sent to the sensing circuit 22 on the substrate 2 via the exposed contact portions 32 and the contact bases 31 of the metal-insert molded contacts 3. In the illustrated second embodiment, there is a high impedance existed among the metal-insert molded contacts 3 to avoid short circuit among the metal-insert molded contacts 3 due to electrical conduction via the body fluids in the patient's digestive tract system. Moreover, there is also a high impedance existed between the metal-insert molded contacts 3 and the substrate 2 to prevent electric current in the sensing circuit 22 on the substrate 2 from leaking via the metal-insert molded contacts 3 to produce electric pulse, which would adversely affect the patient's normal nerve conduction. Further, a layer of metal material can be coated on the metal-insert molded contacts 3, so that the front surfaces 321 of the exposed contact portions 32 exposed from the outer surface 12 of the capsular housing 1 are protected against corrosion.

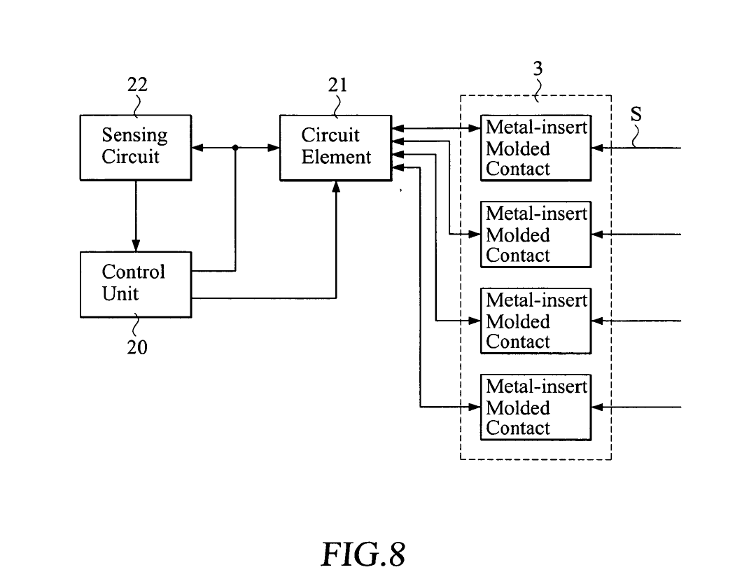

FIG. 8 is a functional block circuit diagram of the present invention. A control unit 20 is electrically connected to the circuit element 21, the sensing circuit 22, and the metal-insert molded contacts 3. The external environmental signals S are sent to the sensing circuit 22 via the metal-insert molded contacts 3 and the circuit element 21. A high impedance exists among the metal-insert molded contacts 3. The control unit 20 may be operated in a signal sensing mode and non-sensing mode. In signal sensing mode, the control unit 20 can receive signals from the metal-insert molded contacts 3 via the circuit element 21 and the sensing circuit 22. The control unit 20 is electrically connected to the metal-insert molded contacts 3, the circuit element 21 and the sensing circuit 22 and is capable of switching a high impedance status between the metal-insert molded contacts 3 and the substrate 2. The impedance between the metal-insert molded contact 3, the substrate 2, and the sensing circuit 22 may be selectively controlled by the control unit 20 via the circuit element 21, depending on signal sensing mode or signal non-sensing mode.

The present invention has been described with some preferred embodiments thereof and it is understood that many changes and modifications in the described embodiments can be carried out without departing from the scope and the spirit of the invention that is intended to be limited only by the appended claims.

What is claimed is:

1. A capsule endoscope, comprising:
a capsular housing defining a sealed inner space-and having an outer surface;
a substrate being arranged in the sealed inner space of the capsular housing at a predetermined position thereof, and mounted with at least an electronic element provided thereon; and
a plurality of metal-insert molded contacts being embedded in the capsular housing at predetermined positions thereof, and each of the metal-insert molded contacts including:
a contact base having a connecting section, which is radially extended into the sealed inner space of the capsular housing to electrically connect to the substrate; and
an exposed contact portion being formed on the contact base and having a front surface exposed from the outer surface of the capsular housing;
whereby when the capsular housing is held in a capsule holder, the exposed contact portions are in contact with a signal contact correspondingly provided on the capsule holder.

2. The capsule endoscope as claimed in claim 1, further comprising a control unit electrically connected to the metal-insert molded contacts to switch a high impedance status between the metal-insert molded contacts and the substrate.

3. The capsule endoscope as claimed in claim 1, wherein the metal-insert molded contacts each have a layer of metal material coated thereon.

4. The capsule endoscope as claimed in claim 1, wherein the contact bases of the metal-insert molded contacts each are an L-shaped sheet member.

5. The capsule endoscope as claimed in claim 1, wherein the contact bases of the metal-insert molded contacts each are a flexible circuit board.

6. The capsule endoscope as claimed in claim 1, wherein the exposed contact portions of the metal-insert molded contacts are formed on the contact bases by way of stamping.

7. The capsule endoscope as claimed in claim 1, wherein the exposed contact portions of the metal-insert molded contacts are formed on the contact bases by way of riveting.

8. The capsule endoscope as claimed in claim 1, wherein the exposed front surfaces of the exposed contact portions are flush with the outer surface of the capsular housing.

9. A capsule endoscope, comprising:
a capsular housing defining a sealed inner space and having an outer surface;
a substrate being arranged in the sealed inner space of the capsular housing at a predetermined position thereof, and having a sensing circuit provided thereon; and
a plurality of metal-insert molded contacts being embedded in the capsular housing at predetermined positions thereof, and each of the metal-insert molded contacts including:
a contact base having a connecting section, which is radially extended into the sealed inner space of the capsular housing to electrically connect to the sensing circuit on the substrate; and
an exposed contact portion being formed on the contact base and having a front surface exposed from the outer surface of the capsular housing;
wherein there is a high impedance existed among the metal-insert molded contacts; and the metal-insert molded contacts contact and sense external environmental signals around a position at where the capsular housing is located; and the sensed external environmental signals are sent to the sensing circuit on the substrate via the exposed contact portions and the contact bases of the metal-insert molded contacts.

10. The capsule endoscope as claimed in claim 9, further comprising a control unit electrically connected to the metal-insert molded contacts to switch a high impedance status that existed between the metal-insert molded contacts and the substrate.

11. The capsule endoscope as claimed in claim 9, wherein the metal-insert molded contacts each have a layer of metal material coated thereon.

12. The capsule endoscope as claimed in claim 9, wherein the contact bases of the metal-insert molded contacts each are an L-shaped sheet member.

13. The capsule endoscope as claimed in claim 9, wherein the contact bases of the metal-insert molded contacts each are a flexible circuit board.

14. The capsule endoscope as claimed in claim 9, wherein the exposed contact portions of the metal-insert molded contacts are formed on the contact bases by way of stamping.

15. The capsule endoscope as claimed in claim 9, wherein the exposed contact portions of the metal-insert molded contacts are formed on the contact bases by way of riveting.

16. The capsule endoscope as claimed in claim 9, wherein the exposed front surfaces of the exposed contact portions are flush with the outer surface of the capsular housing.

17. The capsule endoscope as claimed in claim 9, wherein the external environmental signals sensed by the metal-insert molded contacts comprises at least one of a temperature signal, a pressure signal, and a pH signal.
